(19) United States
(12) Patent Application Publication
(10) Pub. No.: US 2026/0317702 A1
(43) Pub. Date: Oct. 15, 2026

(54) COMBINATION TREATMENT OF INDUCED PLURIPOTENT STEM CELLS USING INTERLEUKINS

(71) Applicant: Honed Life Sciences, LLC, Irvine, CA (US)

(72) Inventors: Avinash Seth, Mission Viejo, CA (US); Nikko Lowe, City of Industry, CA (US); Kurt Brillhart, Mission Viejo, CA (US)

(21) Appl. No.: 19/236,144

(22) Filed: Jun. 12, 2025

Related U.S. Application Data
(63) Continuation of application No. 17/392,620, filed on Aug. 3, 2021, now abandoned.
(60) Provisional application No. 63/060,483, filed on Aug. 3, 2020.

(51) Int. Cl. C12N 5/071

(52) U.S. Cl. CPC C12N 5/0602; C12N 2500/62; C12N 2501/2301; C12N 2501/2303; C12N 2501/2306; C12N 2501/2314; C12N 2501/50; C12N 2501/999; C12N 2506/45

(57) ABSTRACT

Induced pluripotent stem cells are treated using a combination of compounds that improve competence of the induced pluripotent stem cells in responding to differentiation signals and/or improve the efficiency of differentiation of the treated induced pluripotent stem cells in differentiation towards a desired phenotype in a synergistic manner. The combination treatment can incorporate two or more of prolongation of early G1 phase, treatment with an interleukin, modulation of DNA methylation, modulation of histone acetylation, and activation of the Wnt pathway. Cells derived from induced pluripotent stem cells so treated can be used in regenerative therapy and production of organoids.

This application claims priority U.S. patent application Ser. No. 17/392,620, filed Aug. 3, 2021, which claims priority to U.S. Provisional Patent Application No. 63/060,483 filed on Aug. 3, 2020. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is compositions and methods for enhancing Induced Pluripotent Stem Cell (iPSC) differentiation.

### BACKGROUND

The following background discussion includes information that may be useful in understanding the present invention. It is not an admission that any of the information provided herein is prior art or relevant to the presently claimed invention, or that any publication specifically or implicitly referenced is prior art.

Induced pluripotent stem cells (IPSCs) have similar properties to embryonic stem cells (Induction of pluripotent stem cells from mouse embryonic and adult fibroblast cultures by defined factors, K. Takahashi and a S. Yamanaka, 126 Cell 663-76, 2006), and therefore may be capable of developing into therapies to treat degenerative disease. It should be appreciated, however, that these induced pluripotent stem cells are distinct from naturally occurring multipotent and pluripotent stem cells found in somatic tissues, such as mesenchymal stem cells. These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

However, several problems have prevented iPSCs from being widely used. For example, the competence of iPSCs in responding to differentiating conditions can be low, resulting in poor yield of differentiated cells. Similarly, controlled differentiation of iPSCs into cells having the desired phenotype has proven challenging.

Attempts have been made to improve the performance of naturally occurring multipotent and pluripotent stem cells collected from somatic tissue in regard to differentiation. Bastidas-Coral et al. (Hindawi Publishing Corporation Stem Cells International Volume 2016, Article ID 1318256) describes the effect of various cytokines on differentiation of naturally occurring stem cells harvested from adipose tissue. Similarly, Lam et al. (Liver Transplantation 16:1195-1206, 2010) describes the effects of IL-6 on differentiation of naturally occurring mesenchymal stem cells. Xie et al. (Stem Cell Research & Therapy (2018) 9:13) describes the effect of IL-6 on differentiation of naturally occurring stem cells collected from bone marrow. Khajeniazi et al. (Biol. Chem. 2016; aop, DOI 10.1515/hsz-2016-0151) describes the effect of IL-6 and of 5-azacytidine on differentiation of naturally occurring mesenchymal stem cells. It is not evident, however, that such approaches are applicable to iPSCs.

Approaches have also been developed to improve differentiation of partially committed cells derived embryonic stem cells and partially committed stem cells derived from iPSCs. Hwang et al. (Scientific Reports 4: 5916 DOI: 10.1038/srep05916) has found that treatment of partially committed progenitor cells derived from embryonic stem cells with WNT3A protein improved their further differentiation towards a myogenic phenotype. Ackermann et al, (Haematologica, 2021; 106(5) 1354-1367) reports that certain interleukins can be used to improve differentiation of partially committed hemato-endothelial progenitor cells derived from iPSCs towards a hematopoietic progenitor phenotype. Sulistio et al. (Mol Neurobiol DOI 10.1007/s12035-017-0594-3) describes the use of interleukins to improve the differentiation of partially committed neural stem cells derived from iPSCs towards a neuron phenotype. Kondo et al. (PLOS ONE August 2014 Volume 9, Issue 8, e104010) describes using a drug commonly used in treating epilepsy (valproic acid) to improve differentiation of hepatic progenitor cells derived from iPSCs towards a hepatic cell phenotype. It is not apparent, however, if such approaches are effective in cells that have not undergone partial differentiation towards the desired cell phenotype.

Thus, there is still a need for methods of improving the competence of induced pluripotent stem cells to respond to differentiating stimulus and/or providing a user with improved methods for directing differentiation of induced pluripotent stem cells towards a desired phenotype.

### SUMMARY OF THE INVENTION

The inventive subject matter provides compositions and methods for enhancing efficiency of differentiation and direction of the differentiation of induced pluripotent stem cells (iPSCs) using an interleukin, and interleukin analog, and/or an interleukin receptor in combination with a compound that increases the duration of early G1 phase in the iPSCs, and in some instances compounds that modulate pathways related to DNA methylation and/or histone acetylation. These factors can be selected to control the developmental path of iPSCs so treated, for example directing differentiation towards hematopoietic, neuronal, hepatic, and/or cardiac cell types. Treated iPSCs are particularly useful for therapeutic purposes and in the construction of differentiated organoids.

### BRIEF DESCRIPTION OF THE DRAWING

FIG. 1 is a schematic of a method according to the inventive subject matter.

### DETAILED DESCRIPTION

The Inventor has found that treatment of induced pluripotent stem cells (iPSCs) with an interleukin, interleukin analog, and/or interleukin receptor in combination with treatment that extends early G1 phase in the iPSCs can result in increased efficiency of differentiation of the iPSCs and/or induce differentiation of the treated iPSCs towards a desired phenotype. In some embodiments phenotype of treated iPSCs can be induced or enhanced by treatment with compounds or proteins that stimulate certain signaling pathways, modulate DNA methylation, and/or modulate histone acetylation or deacetylation. ISPCs treated in this fashion are useful in regenerative medicine, and can be used for the development of differentiated organoids useful as models for disease and/or screening potential therapeutic compounds.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Induced pluripotent stem cells (iPSCs) have the potential, as part of regenerative medicine, to treat many different types of diseases (e.g. Alzheimer's disease, Parkinson's disease, cardiovascular disease, and amyotrophic lateral sclerosis) as well as injuries (e.g., cardiovascular events, burns, trauma, etc.). ISPCs can also be used to generated differentiated organoids, which can provide model systems useful for emulating disease states and/or screening potential modes of treatment. However, one of the difficulties associated with this treatment relatively low efficiency in differentiation of iPSCs into differentiated cells, as well as direction of differentiation towards the desired differentiated phenotype.

Induced pluripotent stem cells can be generated in any suitable fashion. For example, differentiated or partially differentiated cells (e.g. fibroblasts, adipose stromal cells, etc.) can be treated with retroviruses that encode transcription factors that result in de-differentiation and return to a pluripotent stem cell state. Such factors can include the Yamanaka factors (i.e., Oct3/4, Sox2, Klf4, c-Myc) or a subset of these. Such factors can also be introduced by Lentivirus, Sendai virus, and/or Adenovirus vectors. Alternatively, non-viral vectors can be used to induce pluripotence. Such non-viral vectors include liposomes that incorporate encoding mRNA, PiggyBac® transposons, and minicircle vectors. In other embodiments, such protein factors can be incorporated into the cells directly rather than through transcription and/or translation of encoding genes.

It should be appreciated that such iPSCs can be expanded in tissue culture prior to exposure to priming or differentiating stimuli that result in differentiation into one or more desired phenotypes. While iPSCs can be derived from somatic cells obtained from an individual to be treated, it should be appreciated that iPSCs so generated are distinct from naturally occurring pluripotent mesenchymal stem cells obtained from various tissues (e.g. adipose tissue, cardiac tissue, etc.), which have been exposed to a variety of potentially determinative environmental and chemical cues in situ prior to isolation and expansion.

Induced pluripotent stem cells so produced have the ability to differentiate into a variety of cell types, however current methods for inducing such differentiation often show low efficiency. Similarly, current methods are often ineffective in directed differentiation of iPSCs towards the desired phenotype(s) in an efficient manner. The Inventor believes that such issues can be resolved using a combination of approaches, and that such combinations can yield synergistic (i.e., greater than additive effects of the contributions of individual components) in regard to efficiency of inducing differentiation and in differentiation into one or more desired phenotypes.

In some embodiments of the inventive concept, one component of such a combined approach is to treat iPSCs to increase the percentage of iPSCs that are in early G1 phase, which in turn improves their competency to respond to differentiation signals. For example, the competency of iPSCs so treated to respond to a differentiation signal can be increased by 10%, 20%, 30%, 40%, 50%, 60%, 70%, 80%, 90%, 100&, or more than 100% relative to untreated iPSCs. In some embodiments, the percentage of iPSCs that are in early G1 phase can be increased by activating retinoblastoma protein (Rb protein) within the iPSCs. In such embodiments Rb protein can be activated in any suitable fashion. In some of such embodiments Rb protein in iPSCs can be activated by contacting the cells with a compatible organic solvent, such as DMSO (Chetty et al., “A simple tool to improve pluripotent stem cell differentiation” Nat Methods. 2013 June; 10(6): 553-556). For example, DMSO can be applied to iPSCs at a concentration of from 0.1% to 5% for a period of time ranging from 1 hour to 72 hours. Alternatively, Rb protein in iPSCs can be activated by treating the cells with a compound or peptide that complexes with Rb protein and enhances its affinity for E2F transcription factors. Such compounds or peptides can be identified using binding assays known in the art.

In some embodiments of the inventive concept a component of such a combined approach can be an interleukin. Such an interleukin can be an interleukin that is not generated by the iPSCs (i.e., an exogenous interleukin). In some embodiments such an interleukin is a pro-inflammatory interleukin. In other embodiments such an interleukin is an anti-inflammatory interleukin. Such an interleukin can act to increase the iPSCs competence for responding to differentiating factors. In some embodiments, such an interleukin can act as a differentiating factor, improving the efficiency of iPSCs differentiation towards a desired phenotype. In some embodiments a single interleukin can provide both effects. In other embodiments, the interleukin component of the combined approach can be provided as a mixture of two or more interleukins in effective amounts and/or proportions. For example, IL-6, soluble IL-6 receptor (IL-6r), or a hybrid protein that includes components of IL6, IL6r or both IL6 and IL6r can be used to direct iPSCs differentiation towards osteogenic or hepatic cell phenotypes. Similarly, IL-17 can be used to direct iPSCs differentiation towards osteogenic cell phenotypes. In another example, IL-4 can be used to direct iPSCs differentiation towards hematopoietic or osteogenic cell phenotypes. IL-3 or IL-1 (e.g. IL-1β) can be used to direct iPSCs differentiation towards hematopoietic cell phenotypes.

It should be appreciated that the interleukins cited above are exemplary, and that the Inventor believes that a wide range of interleukins can be useful in increasing the competency of iPSCs to respond to differentiating factors and/or improving the efficiency of iPSCs differentiation towards a desired phenotype. Suitable interleukins include, but are not limited to, IL-2, IL-5, IL-7, IL-8, IL-9, IL-10, IL-11, IL-12, IL-13, IL-14, and/or IL-15. Interleukins utilized in methods of the inventive concept can be provided as a single interleukin or as a mixture of two or more interleukins. Such interleukins can be provided at any suitable concentration, for example from 0.1 ng/mL to 10 mg/mL. In embodiments utilizing combinations of two or more interleukins the weight or molar ratio of one interleukin in the mixture to another interleukin in the mixture can range from 1:100 to 100:1. In some embodiments, synergistic (i.e., greater than additive effects of the individual components of the mixture when applied separately) in regard to increasing the competency of iPSCs to respond to differentiating factors and/or improving the efficiency of iPSCs differentiation towards a desired phenotype are observed when a combination of interleukins is used.

In some embodiments, combined treatment of iPSCs can include the use of compounds or environmental factors that modulate iPSCs epigenetics. For example, compounds can be applied to iPSCs that modulate DNA methylation and/or histone acetylation. For example, in some embodiments of the inventive concept a compound that inhibits histone deacetylation can be applied to iPSCs, which can in turn promote differentiation of treated iPSCs towards hepatic cell phenotypes. Suitable histone deacetylation inhibitors include bromides, vorinostat, panobinostat, belinostat, and valproic acid. In other embodiments, iPSCs can be treated with a compound that inhibit DNA methyltransferase (e.g. 5-asacytidine) in order to reduce DNA methylation.

In some embodiments of the inventive concept, compounds that are included in combination treatment of iPSCs can be selected on the basis of their action on certain regulatory pathways within iPSCs. For example, as noted above modulation of regulatory pathways that can be manipulated by activating Rb protein can generally act to prolong early G1 phase in iPSCs. The same regulatory pathways can also modulate DNA methylation and histone acetylation-providing a combined effect. Similar, differentiation of iPSCs towards a myogenic phenotype can be improved by activation of the Wnt pathway (for example, by application of exogenous WNT3A protein to iPSCs).

In methods of the inventive concept, two or more of the above cited approaches are applied in combination to iPSCs in order to improve their responsiveness to differentiating signals and/or improve the efficiency of differentiation of the iPSCs towards a desired phenotype. For example, Rb protein activation (e.g., by application of DMSO) can be combined with application of one or more interleukins (e.g. IL-6, IL-1β, IL-3, and/or IL-17). Alternatively, one or both of these approaches can be combined with application of a compound that modulates DNA methylation and/or histone acetylation (e.g., a histone deacetylation inhibitor) to the iPSCs, or (or in addition to) application of a compound that modulates a Wnt pathway of the iPSCs (e.g. WNT3A protein). The Inventor believes that such a combined approach can provide a synergistic (i.e., greater than the additive contribution of individual components when applied individually) on improving iPSCs competence in responding to differentiation signals and/or improving efficiency of differentiation of iPSCs towards a desired cell phenotype.

In some of such combined therapies two or more components of the therapy can be applied to iPSCs simultaneously. In other embodiments a first subset of the components of the combined approach can be applied initially, followed by application of a second subset of the components of the combined approach. Such first and second subsets of components are distinct from one another, but in some embodiments can include overlapping components. For example, in some embodiments DMSO and IL-6 can be provided to iPSCs in combination initially, followed by a combination of DMSO, IL-6, one or more other interleukins, and/or a histone acetylation inhibitor and/or a Wnt pathway activating compound. Alternatively, DMSO, IL-6, a non-IL-6 interleukin, a histone acetylation inhibitor, and a Wnt pathway activating compound can be administered to iPSCs simultaneously.

Components of the combination treatment of iPSCs can be applied to iPSCs on any suitable schedule. For example, components of the combination treatment can be applied to iPSCs continuously for up to 28 days, or periodically. When applied periodically the time between applications can range from 5 minutes to one week.

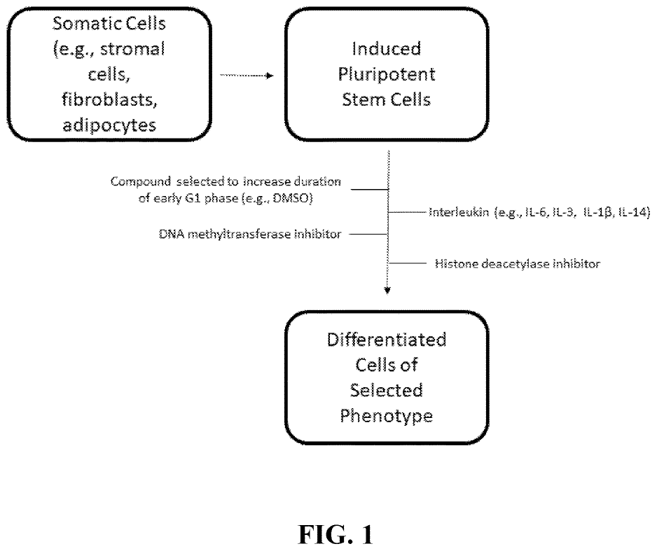

An example of a combination treatment method of the inventive concept is shown schematically in FIG. 1. Typical steps are as follows:
(1) Induced pluripotent stem cells (iPSCs) are generated from differentiated or partially differentiated cells (e.g. stromal cells, fibroblasts, adipocytes). If the intended use is for regenerative medicine such cells are preferably obtained from the individual in need of treatment. If necessary iPSCs can be expanded in culture prior to combination treatment.
(2) The cultured IPSCs are incubated with two or more of the components of the combination treatment as described above.
(3) If intended for use in regenerative medicine, after the incubation, the differentiated or differentiating cells so produced can be collected. If intended for use in generating differentiated organoids, the differentiated or differentiating cells can be allowed to form such organoids spontaneously or can be otherwise treated to encourage organoid formation.

Cells resulting from combination treatment of IPSCs can be applied to an individual in need of treatment with stem cell therapy. For example, cells resulting from combination treatment of iPSCs can be applied systemically (e.g., by injection and/or infusion) or locally at the site where the treatment is needed (by injection, topical application, application of a medical device incorporated the primed iPSCs, etc.).

As noted above, cells resulting from combination treatment of iPSCs can be provided in a form suitable for injection or infusion. In such embodiments primed IPSCs in a pharmaceutically acceptable carrier that provides support for the cells resulting from combination treatment of iPSCs as well as being compatible with the individual in need of treatment. Such pharmaceutically compatible carriers can, for example, include salts, pH buffers, glucose/dextrose, and/or isotonic agents. In some embodiments such a pharmaceutical carrier can include a secondary pharmaceutical agent. Such a secondary pharmaceutical agent can be directed towards controlling potential side effects of therapy with cells resulting from combination treatment of iPSCs. Alternatively, or in addition, such a secondary pharmaceutical agent can provide complementary therapy for the condition being treated. In some embodiments a synergistic (i.e., greater than additive effect) is provided by the combination of the cells resulting from combination treatment of iPSCs and the secondary pharmaceutical. Suitable secondary pharmaceutical include, but are not limited to, a steroid, a non-steroidal anti-inflammatory drug, an anti-pruritics, a pain control medication, an antibiotic, an ant-viral drug, an anti-fungal drug, and an anti-cancer drug).

In some embodiments, cells resulting from combination treatment of iPSCs are provided as part of a medical appliance. Suitable medical appliances include dressings, patches, implants, and tissue scaffolds. In such embodiments cells resulting from combination treatment of iPSCs can be provided in a suspension (e.g., a gel or thickened fluid) that coats or is integrated into the medical appliance. In other embodiments cells resulting from combination treatment of iPSCs can be attached (e.g., through noncovalent interactions) to a surface of the biomedical appliance. In some embodiments IPSCs can be provided within a porous medical appliance, where the pores are sized to allow IPSCs to migrate from the interior of the medical appliance following implantation.

Another embodiment of the inventive concept is a method of treating an individual in need of treatment with iPSCs. In some embodiments differentiated or partially differentiated stem cells used to produce IPSCs can be obtained from the individual to be treated. In other embodiments IPSCs to be primed can be derived from another individual or cell culture. The differentiated and/or differentiating cells provided by combination treatment of such iPSCs can be applied to an individual in need of treatment as described above.

Such an individual to be treated can be treated for a disease or condition (e.g., cardiovascular disease, arthritis, Alzheimer's disease, Parkinson disease, amyotrophic lateral sclerosis, diabetic retinopathy, macular degeneration, retinitis pigmentosa, damage due to age, etc.) or injury (e.g., accidental injury, surgical injury, second, third, or fourth degree burns, brain damage due to stroke, concussion, etc.). Such individuals can be treated with cells resulting from combination treatment of iPSCs and administered as described above in any effective number and on any effective schedule. For example, from 10² to 10⁸ cells resulting from combination treatment of iPSCs can be provided by systemic and/or local application as described above. Such cells resulting from combination treatment of iPSCs can be provided as a single bolus, or as repeated administrations over a period of time. In some embodiments cells resulting from combination treatment of iPSCs can be initially provided in systemically, followed by additional local treatment. Alternatively, primed IPSCs can be initially provided locally (e.g., at a surgical site during or following a surgical procedure) followed by systemic delivery. In other embodiments primed IPSCs can be provided systemically and locally over the same period of time.

Another embodiment of the inventive concept is a method for generating differentiated organoids. Such differentiated organoids can, for example, provide functional hematopoietic tissue that can act as a source of blood or immune cells, provide functional models or organs (e.g., heart, liver, skeletal muscle, nervous system, etc.) that are useful for understanding disease processes and for screening for effective therapies. In some embodiments such differentiated organoids can arise from self-organization of cells resulting from combination treatment of iPSCs in culture. In other embodiments cells resulting from combination treatment of iPSCs can be applied to specific locations on a surface (such as a plate or a biocompatible scaffold) to provide the desired geometry, for example by droplet dispensing of desired cell types to desired areas. In other embodiments a differentiated organoid can be produced by 3D printing of cells resulting from combination treatment of iPSCs, for example as a suspension of such cells in a polymerizable pre-polymer followed by initiation of polymerization (e.g., by irradiation).

Alternatively, iPSCs can be printed or applied to a scaffold as described above, then subjected to combination treatment in order to provide differentiated cells in situ. In such embodiments iPSCs can be expanded following printing or application to a scaffold in order to increase their number or density prior to application of combination treatment. For example, iPSCs can be distributed on a tissue scaffold and allowed to grow to confluence or partial confluence prior to application of combination treatment, thereby providing confluent or partially confluent differentiated cells within the organoid.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A method of generating differentiated cells from induced pluripotent stem cells (iPSCs), comprising:
obtaining a plurality of iPSCs;
contacting the plurality of iPSCs with a first amount of a compound selected to increase duration of early G1 phase in the plurality of iPSCs; and
contacting the plurality of iPSCs with a second amount of an exogenous interleukin,
wherein the first amount and second amount are effective to provide a synergistic effect to improve the competence or increase efficiency of iPSCs to undergo differentiation that exceeds the additive effects of the first amount of the compound selected to increase duration of early G1 phase and the second amount of the exogenous interleukin.

2. The method of claim 1, wherein the compound selected to increase duration of early G1 phase in the plurality of iPSCs is an organic solvent.

3. The method of claim 2, wherein the organic solvent is dimethyl sulfoxide.

4. The method of claim 1, wherein the exogenous interleukin is selected to induce differentiation of the plurality of iPSCs toward a pre-selected phenotype.

5. The method of claim 4, wherein the pre-selected phenotype is selected from the group consisting of a blood cell, an immune cell, a hepatic cell, a muscle cell, a cardiac cell, and a neuron.

6. The method of claim 1, wherein the exogenous interleukin is selected from the group consisting of IL-6, IL-3, IL-1β, and IL-14.

7. The method of claim 1, comprising contacting the plurality of iPSCs with a supplementary interleukin.

8. The method of claim 1, comprising contacting the plurality of iPSCs with a soluble IL-6 receptor protein or a hybrid protein comprising at least a portion of IL-6 and at least a portion of IL-6 receptor protein, or a complex comprising IL-6 and IL-6 receptor protein.

9. The method of claim 1, comprising contacting the plurality of iPSCs with an inhibitor of DNA methyltransferase.

10. The method of claim 1, comprising contacting the plurality of iPSCs with an inhibitor of histone deacetylation.

11. The method of claim 1, comprising contacting the plurality of iPSCs with a compound selected to activate a Wnt pathway of the plurality of iPSCs.

12. A composition, comprising:
a plurality of iPSCs;
a first amount of a compound selected to increase duration of early G1 phase in the plurality of iPSCs; and
a second amount of an exogenous interleukin,
wherein the first amount and second amount are effective to provide a synergistic effect to improve the competence or increase efficiency of iPSCs to undergo differentiation that exceeds the additive effects of the first amount of the compound selected to increase duration of early G1 phase and the second amount of the exogenous interleukin.

13. The composition of claim 12, wherein the compound selected to increase duration of early G1 phase in the plurality of iPSCs is an organic solvent.

14. The composition of claim 13, wherein the organic solvent is dimethyl sulfoxide.

15. The composition of claim 12, wherein the exogenous interleukin is selected to induce differentiation of the plurality of iPSCs toward a pre-selected phenotype.

16. The composition of claim 15, wherein the pre-selected phenotype is selected from the group consisting of a blood cell, an immune cell, a hepatic cell, a muscle cell, a cardiac cell, and a neuron.

17. The composition of claim 12, wherein the exogenous interleukin is selected from the group consisting of IL-6, IL-3, IL-1β, and IL-14.

18. The composition of claim 12 further, comprising a supplementary exogenous interleukin.

19. The composition of claim 12, further comprising IL-6 receptor protein or a fragment thereof.

20. The composition of claim 12, further comprising an inhibitor of DNA methyltransferase.

21. The composition of claim 12, further comprising an inhibitor of histone deacetylation.

22. The composition of claim 12, further comprising a compound selected to activate a Wnt pathway of the plurality of iPSCs.
